(19) United States
(12) Patent Application Publication
(10) Pub. No.: US 2021/0330818 A1
(43) Pub. Date: Oct. 28, 2021

(54) METHOD FOR ANALYZING NON-OBSERVED EFFECT CONCENTRATION (NOEC) OF CHEMICAL ON ORGANISM

(71) Applicant: Zhejiang Academy of Agricultural Sciences, Hangzhou City (CN)

(72) Inventors: Wen Song, Hangzhou City (CN); Changxing Wu, Hangzhou City (CN); Xinxin Zhou, Hangzhou City (CN); Liping Chen, Hangzhou City (CN); Tao Cang, Hangzhou City (CN); Mingfei Xu, Hangzhou City (CN); Yi Zhang, Hangzhou City (CN)

(21) Appl. No.: 17/016,585

(22) Filed: Sep. 10, 2020

(30) Foreign Application Priority Data
Apr. 23, 2020 (CN) 202010326649.8

(51) Int. Cl. A61K 49/00

(52) U.S. Cl. CPC A61K 49/0004

(57) ABSTRACT

The present invention provides a method for analyzing the non-observed effect concentration (NOEC) of a chemical on an organism. The analysis method includes the following steps: 1) conducting a chronic toxicity test on a test organism with a test chemical at different concentrations, and conducting assays to obtain several sets of endpoint effect data; 2) classifying the several sets of endpoint effect data obtained in step 1); and 3) constructing hypothesis testing models with the data classified in step 2), and according to the statistical significance values from hypothesis testing models, among the same set of endpoint effect data, adopting the highest concentration of the test chemical that do not produce a significant effect as NOEC within the set; and among the different sets of endpoint effect data, adopting NOEC of the set with the smallest NOEC value as NOEC of the test chemical on the test organism.

### CROSS REFERENCE TO RELATED APPLICATIONS

This application claims priority, and benefit under 35 U.S.C. § 119(e) of Chinese Patent Application No. 202010326649.8 filed 23 Apr. 2020. The disclosure of the prior application is hereby incorporated by reference as if fully set forth below.

### TECHNICAL FIELD

The present invention belongs to the technical field of data analysis for agrochemicals, and in particular relates to a method for analyzing the non-observed effect concentration (NOEC) of a chemical on an organism.

### BACKGROUND

Studying the toxic effect of a chemical is a necessary means to predict the safe exposure limit of a chemical, and non-observed effect concentration (NOEC) is one of the important parameters. NOEC refers to the highest concentration of a test substance that exhibits no significant effect on a test organism within a certain period of time compared to the control. This indicator is extremely important for evaluating the chronic toxicity of a chemical, and is an indispensable basis for formulating hygiene standards for a chemical. Since NOEC is very close to a threshold dose (a minimum dose that causes adverse effects), it requires reliable test data and sensitive and accurate statistical methods to support data analysis. In the prior art, significance analysis of difference and multiple comparisons (commonly Dunnett's t test) are commonly used to determine whether the concentration of a treatment group is NOEC by comparing the significance of the difference between the average value of the treatment group and the average value of a control group; and EC1 (1% effective inhibitory concentration) is also used as an NOEC threshold. The above methods are either simple or rough, or lack of consideration for the properties of data, thereby ignoring the impact of data type, monotonicity, normality and homogeneity of variance on the applicability of the analysis method. The blind application of parametric statistical methods cannot guarantee the efficiency of the statistical analysis and the accuracy of results.

Therefore, in view of the shortcomings of the prior art, there is an urgent need to develop a statistical strategy based on the determination of properties of the test data for the effective analysis of NOEC.

### SUMMARY

In view of this, the present invention is intended to provide a method for analyzing NOEC of a chemical on an organism; and the analysis method can ensure the efficiency and accuracy of the analysis.

The present invention provides a method for analyzing the NOEC of a chemical on an organism, including the following steps:

1) conducting a chronic toxicity test on a test organism with a test chemical at different concentrations, and conducting assays to obtain several sets of endpoint effect data;

2) classifying the several sets of endpoint effect data obtained in step 1) into the following types: a, b, c, d and e, where, a: the data have monotonicity; b: the data are binary variables that do not have monotonicity; c: the data are continuous variables that do not have monotonicity, and the data conform to the normal distribution and homogeneity of variance; d: the data are continuous variables that do not have monotonicity, but the data only conform to the normal distribution; and e: the data are continuous variables that do not have monotonicity, but the data do not conform to the normal distribution; and

3) constructing hypothesis testing models with the data classified in step 2), and according to the statistical significance values from hypothesis testing models, among the same set of endpoint effect data, adopting the highest concentration of the test chemical that do not produce a significant effect as NOEC within the set; and among the different sets of endpoint effect data, adopting NOEC of the set with the smallest NOEC value as NOEC of the test chemical on the test organism;

where, a trend test model is adopted when the data are consistent with a;

a non-parametric paired comparison test model is adopted when the data are consistent with b;

a paired comparison test model is adopted when the data are consistent with c;

a heteroscedasticity paired comparison test model is adopted when the data are consistent with d; and

a non-parametric paired comparison test model is adopted when the data are consistent with e.

Preferably, the test organism includes animals.

Preferably, the animals include insects and birds.

Preferably, when the test organism is Trichogramma, the endpoint effect data include one or more of egg yield, emergence rate, adult survival time, parasitism rate, mortality rate and hatching rate.

Preferably, when the test organism is Trichogramma, the action of the test chemical on Trichogramma in step 1) is achieved by the egg card-dipping method.

Preferably, when the test organism is quail, the endpoint effect data include one or more of 14-day survival rate, embryo survival rate, hatching rate, emergence rate, feeding amount, body weight, average daily egg production, average egg production and stillbirth rate.

Preferably, when the test organism is quail, the action of the test chemical on the quail in step 1) is achieved by feeding the quail with a feedstuff admixed with the test chemical.

Preferably, the different concentrations in step 1) include 4 to 10 different concentrations.

Preferably, the trend test model includes Jonckheere-Terpstra test; the non-parametric paired comparison test model includes Fisher's exact test based on Bonferroni-Holm correction; the paired comparison test model includes Dunnett's test; the heteroscedasticity paired comparison test model includes Tamhane-Dunnett test; and the non-parametric paired comparison test model includes Mann-Whitney test based on Bonferroni-Holm correction.

The present invention has the following beneficial effects: The method for analyzing NOEC of a chemical on an organism provided by the present invention can ensure the efficiency and accuracy of the analysis by classifying the endpoint effect data of a chronic toxicity test and using different trend test models for different types of data.

### BRIEF DESCRIPTION OF DRAWINGS

FIG. 1 is a flow chart for the NOEC analysis method according to the present invention.

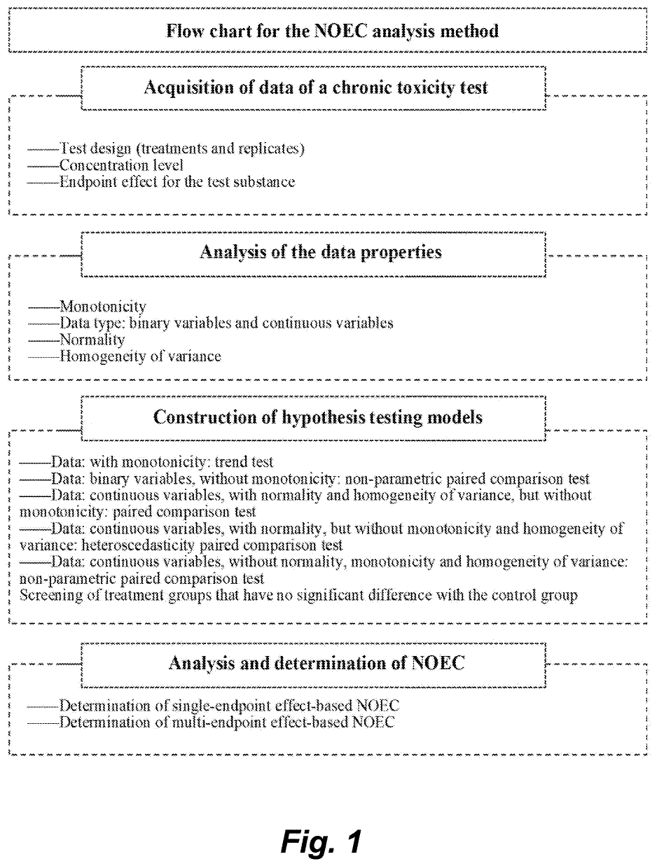

### DETAILED DESCRIPTION

The present invention provides a method for analyzing NOEC of a chemical on an organism, including the following steps:

1) conducting a chronic toxicity test on a test organism with a test chemical at different concentrations, and conducting assays to obtain several sets of endpoint effect data;

2) classifying the several sets of endpoint effect data obtained in step 1) into the following types: a, b, c, d and e, where, a: the data have monotonicity; b: the data are binary variables that do not have monotonicity; c: the data are continuous variables that do not have monotonicity, and the data conform to the normal distribution and homogeneity of variance; d: the data are continuous variables that do not have monotonicity, but the data only conform to the normal distribution; and e: the data are continuous variables that do not have monotonicity, but the data do not conform to the normal distribution; and

3) constructing hypothesis testing models with the data classified in step 2), and according to the statistical significance values from hypothesis testing models, among the same set of endpoint effect data, adopting the highest concentration of the test chemical that do not produce a significant effect as NOEC within the set; and among the different sets of endpoint effect data, adopting NOEC of the set with the smallest NOEC value as NOEC of the test chemical on the test organism. A trend test model is adopted when the data are consistent with a; a non-parametric paired comparison test model is adopted when the data are consistent with b; a paired comparison test model is adopted when the data are consistent with c; a heteroscedasticity paired comparison test model is adopted when the data are consistent with d; and a non-parametric paired comparison test model is adopted when the data are consistent with e.

In the present invention, a chronic toxicity test is conducted on a test organism with a test chemical at different concentrations, and several sets of endpoint effect data are obtained by assays. The present invention has no special limitation on the type of the test chemical, and any type of chemical may be used. The test chemical may be a single compound or a multi-component chemical substance. In a specific implementation of the present invention, the chemical is usually an agrochemical, such as dimethoate, imidacloprid, acetamiprid and MCPA. In the present invention, the different concentrations preferably include 4 to 10 different concentrations, more preferably include 6 different concentrations, and further more preferably include one blank control and 5 different concentrations of the test chemical. The present invention has no special limitation on the difference among or the ratio of the different concentrations set for the test chemical, and the equal difference and equal ratio settings may be adopted, or the irregular difference setting may also be adopted. The present invention has no special limitation on the type of the test organism, and the test organism is preferably an animal, and more preferably includes insects and birds. In a specific implementation of the present invention, Trichogramma and quail are adopted as examples.

In the present invention, when the test organism is Trichogramma, the endpoint effect data include one or more of egg yield, emergence rate, adult survival time, parasitism rate, mortality rate and hatching rate. In a specific implementation of the present invention, the endpoint effect data include egg yield, emergence rate and adult survival time. In the present invention, when the test organism is Trichogramma, dimethoate and imidacloprid are adopted, by way of example, as the test chemicals; and the action of the test chemical on Trichogramma is preferably achieved by the egg card-dipping method. The present invention has no special limitation on the specific operations for the egg card-dipping method, a conventional egg card-dipping method in the art may be used, and the detailed steps are described in the examples.

In the present invention, when the test organism is quail, the endpoint effect data include one or more of 14-day survival rate, embryo survival rate, hatching rate, emergence rate, feeding amount, body weight, average daily egg production, average egg production and stillbirth rate. In a specific implementation of the present invention, the endpoint effect data include body weight, average daily egg production and stillbirth rate. In the present invention, when the test organism is quail, acetamiprid and MCPA are adopted, by way of example, as test chemicals; and the action of the test chemical on the quail is achieved by feeding the quail with a feedstuff admixed with the test chemical. In the present invention, preferably, the quail is fed with a feedstuff admixed with the test chemical for 40 to 48 days, and body weight, average daily egg production, average egg production and stillbirth rate are recorded.

In the present invention, after several sets of endpoint effect data are obtained, the several sets of endpoint effect data are classified into the following types: a: the data have monotonicity; b: the data are binary variables that do not have monotonicity; c: the data are continuous variables that do not have monotonicity, and the data conform to the normal distribution and homogeneity of variance; d: the data are continuous variables that do not have monotonicity, but the data only conform to the normal distribution; and e: the data are continuous variables that do not have monotonicity, but the data do not conform to the normal distribution. In the present invention, preferably, the endpoint effect data are analyzed and then classified; the analysis includes analysis on data type, monotonicity, normality, and homogeneity of variance; the analysis is conducted by an analysis method in the prior art; the data type includes the binary variable type and the continuous variable type; the monotonicity is visually determined through a scatter plot of dose-response relationship; the data normality is determined by Shapiro-Wilk Test W; and the homogeneity of variance is determined by Levene test.

In the present invention, after the endpoint effect data are classified, hypothesis testing models are constructed with the data classified, and according to the statistical significance values from hypothesis testing models, among the same set of endpoint effect data, the highest concentration of the test chemical that do not produce a significant effect is adopted as NOEC within the set; and among the different sets of endpoint effect data, NOEC of the set with the smallest NOEC value is adopted as NOEC of the test chemical on the test organism.

In the present invention, a trend test model is adopted when the data are consistent with a; a non-parametric paired comparison test model is adopted when the data are consistent with b; a paired comparison test model is adopted when the data are consistent with c; a heteroscedasticity paired comparison test model is adopted when the data are consistent with d; and a non-parametric paired comparison test model is adopted when the data are consistent with e. In the present invention, the trend test model preferably includes Jonckheere-Terpstra test; the non-parametric paired comparison test model preferably includes Fisher's exact test based on Bonferroni-Holm correction; the paired comparison test model preferably includes Dunnett's test; the heteroscedasticity paired comparison test model preferably includes Tamhane-Dunnett test; and the non-parametric paired comparison test model preferably includes Mann-Whitney test based on Bonferroni-Holm correction.

The technical solutions provided by the present invention will be described in detail below with reference to examples, but the examples should not be construed as limiting the claimed scope of the present invention.

### Example 1

Determination of NOEC of Dimethoate and Imidacloprid on Trichogramma

Step 1: Acquisition of Data of a Chronic Toxicity Test

Data of a chronic toxicity test were acquired for a chemical. The data involved data of test design (treatments and replicates), concentration level, endpoint effect for the test chemical and the like.

Trichogramma is one of the natural enemies that are most widely used and have dominant influence. As an important means to control insect pests, chemical pesticides can also cause toxic and side effects to natural enemies of the insect pests that will be killed by the chemical pesticides. The safety of pesticides against natural enemies was verified by conducting a chronic toxicity test of pesticides on Trichogramma ostriniae. In the test, Trichogramma was adopted as a test organism, dimethoate and imidacloprid were adopted as test chemicals, and endpoint effects were egg yield, emergence rate and adult survival time. 6 treatment concentrations (including 1 blank control and 5 test chemical concentrations, see Table 1 for details) were set for each test chemical group.

The egg card-dipping method was adopted. A 1.0 cm×2.0 cm card of rice moth eggs (approximately 100 eggs) was placed in each finger tube, then about 20 Trichogramma adults at 4 h to 6 h after emergence were introduced, and the Trichogramma adults were removed after they had parasitized the rice moth eggs for 24 h. 144 h later, the egg cards were dipped in test solutions with different concentrations separately for 5 s, then taken out and air dried, and put into finger tubes. The tubes were sealed with a black cloth, and then put in an incubator until the emergence of Trichogramma adults was completed. A 0.1% Triton X-100 aqueous solution was adopted as a blank control. 9 replicates were set for each treatment, and divided into three groups for the observation of 3 endpoint effects respectively, namely, with 3 replicates for each endpoint effect. A first group was used to investigate the egg yield; a second group was used to investigate the number of emerged adults, and the emergence rate was calculated (emergence rate=the number of emerged adults/the number of unemerged eggs×100%); and a third group was used to investigate the adult survival time. If the number of emerged adults is less than 30 or the emerged adults have a low vitality and thus cannot be transferred to a new finger tube autonomously, the parasitism rate and survival time will not be investigated.

Step 2: Analysis of the Data Properties

The data type, monotonicity, normality and homogeneity of variance were analyzed separately for the endpoint effect data according to existing techniques to determine the properties of the data. The data type involved the binary variable type and the continuous variable type; the monotonicity was visually determined through a scatter plot of dose-response relationship; the data normality was determined by Shapiro-Wilk Test W; and the homogeneity of variance was determined by Levene test.

The data can be divided into the following five types based on data properties: (1) a first type: the data have monotonicity; (2) a second type: the data are binary variables that do not have monotonicity; (3) a third type: the data are continuous variables that do not have monotonicity, and the data conform to the normal distribution and homogeneity of variance; (4) a fourth type: the data are continuous variables that do not have monotonicity, but the data only conform to the normal distribution; and (5) a fifth type: the data are continuous variables that do not have monotonicity, but the data do not conform to the normal distribution.

In this example, for the dimethoate test group, the egg yield data were consistent with the first type: continuous variables, with monotonicity; the emergence rate data were consistent with the second type: binary variables, without monotonicity; and the adult survival time data were consistent with the third type: continuous variables, with normality and homogeneity of variance, but without monotonicity.

For the imidacloprid test group, the egg yield data were consistent with the fourth type: continuous variables, with normality, but without monotonicity and homogeneity of variance; the emergence rate data were consistent with the first type: binary variables, with monotonicity; and the adult survival time data were consistent with the fifth type: continuous variables, without monotonicity and normality. The above data properties were determined to establish an analysis method.

Step 3: Construction of a Hypothesis Testing Model for NOEC Analysis Based on the Data Properties and Screening of Treatment Groups that had No Significant Difference with the Control Group

The analysis of NOEC can be regarded as a process for proving the existence of poison effect. In essence, unless sufficient evidence can be provided by the data to prove the existence of toxicity, the test substance is assumed to be non-toxic. The hypothesis testing model can not only assess the toxicity of the test substance through the overall characteristics hypothesis and the sample statistical inference, but also provide abundant parametric or non-parametric test solutions for various data types. Even if the dependent variable data do not have monotonicity, and have undeterminable distribution or do not conform to the normality and homogeneity of variance hypothesis, the model still has an applicable solution. In the prior art, a parameter method (commonly Dunnett's t test) is commonly adopted to determine whether the concentration of a treatment group is NOEC by comparing the significance of the difference between the average value of the treatment group and the average value of the control group, which ignores the dependent variable data type, monotonicity and data distribution, and thus cannot guarantee the statistical power and the biological significance of the results. The hypothesis testing model based on the determination of data properties can avoid the blindness of existing methods, which infers the dose-response relationship for the test substance on the premise that a comprehensive consideration is given to data properties and statistical properties of a method.

The hypothesis testing model had the following specific steps:

A trend test model was adopted when the data were consistent with the first type. For the egg yield data of the dimethoate test group and the emergence rate data of the imidacloprid test group, Jonckheere-Terpstra test was adopted.

A non-parametric paired comparison test model was adopted when the data were consistent with the second type. For the emergence rate data of the dimethoate test group, Fisher's exact test based on Bonferroni-Holm correction was adopted.

A paired comparison test model was adopted when the data were consistent with the third type. For the adult survival time data of the dimethoate test group, Dunnett's test was adopted.

A heteroscedasticity paired comparison test model was adopted when the data were consistent with the fourth type. For the egg yield data of the imidacloprid test group, Tamhane-Dunnett test was adopted.

A non-parametric paired comparison test model was adopted when the data were consistent with the fifth type. For the adult survival time data of the imidacloprid test group, Mann-Whitney test based on Bonferroni-Holm correction was adopted.

The above test models were inspected with SPSS software, and the effort involving Bonferroni-Holm correction was done by analysts themselves.

With the above models, the NOEC revealed by each endpoint effect was determined according to the statistical significance value from each test model. For dimethoate, NOEC for chronic toxicity in Trichogramma was calculated as 100 mg/L based on the egg yield; NOEC for chronic toxicity in Trichogramma was calculated as 200 mg/L based on the emergence rate; and NOEC for chronic toxicity in Trichogramma was calculated as 400 mg/L based on the adult survival time. For imidacloprid, NOEC for chronic toxicity in Trichogramma was calculated as 80 mg/L based on the egg yield; NOEC for chronic toxicity in Trichogramma was calculated as 40 mg/L based on the emergence rate; and NOEC for chronic toxicity in Trichogramma was calculated as 160 mg/L based on the adult survival time.

TABLE 1
The effect of dimethoate and imidacloprid on the egg yield,
emergence rate and adult survival time of Trichogramma

| | Mass | Egg | Emergence | Adult |
|---|---|---|---|---|
| | concentration | yield | rate | survival time |
| Chemical | (mg/L) | (eggs) | (%) | (d) |
| Dimethoate | 0 | 37.3 a | 87.81 a | 2.98 a |
| | 25 | 36.1 a | 92.80 a | 3.15 a |
| | 50 | 34.2 a | 93.71 a | 3.27 a |
| | 100 | 31.6 a | 95.44 a | 3.30 a |
| | 200 | 27.2 b | 93.72 a | 2.67 a |
| | 400 | 20.7 b | 78.93 b | 2.32 a |
| Imidacloprid | 0 | 36.4 a | 91.73 a | 3.32 a |
| | 10 | 37.0 a | 90.57 a | 3.60 a |
| | 20 | 37.3 a | 87.81 a | 3.23 a |
| | 40 | 36.0 a | 78.19 a | 3.50 a |
| | 80 | 34.2 a | 56.12 b | 3.21 a |
| | 160 | 28.5 b | 27.65 c | 3.12 a |

Step 4: Analysis and determination of NOEC According to the statistical test results, based on a single set of endpoint effects, the highest concentration of the test substance that did not produce a significant effect was determined as NOEC within the set; based on a comprehensive evaluation of multiple sets of endpoint effects, the smallest NOEC value among multiple sets of endpoint effect-based NOECs was determined as NOEC for the test substance.

Given the results of the test and analysis for each endpoint effect at the test concentrations, NOEC of dimethoate was determined as 100 mg/L; and NOEC of imidacloprid was determined as 40 mg/L.

Validation: The test was conducted once again with dimethoate at a concentration of 90 mg/L and a blank control, the egg yield was investigated for Trichogramma, and there was no significant difference between the two groups.

The test was conducted once again with imidacloprid at a concentration of 35 mg/L and a blank control, the emergence rate was investigated for Trichogramma, and there was no significant difference between the two groups.

### Example 2

Step 1: Acquisition of Data of a Chronic Toxicity Test The chronic toxicity (growth and reproduction) test for quails was adopted as an example. The widespread use of pesticides in agricultural production has produced a tremendous impact on birds that mainly look for food on farmland. The exposure to low-dose or slightly-toxic pesticides will not cause the death of birds, but will affect the growth, reproduction and other behaviors of birds. By evaluating the results of a toxicity test for birds and other model organisms for environmental toxicology, combining the field exposure level, and extrapolating to the wild environment, the environmental risks of a pesticide can be more fully understood.

In the test, the quail was adopted as a test organism, acetamiprid and MCPA were adopted as test chemicals, and the end effects were body weight, average daily egg production and stillbirth rate. 6 treatment concentrations (including 1 blank control and 5 test chemical concentrations, see Table 2 for details) were set for each test chemical group.

Healthy and lively quails, which were 30-day old and weighed 90 g to 110 g, were selected for the test. The male and female were raised separately, with 10 quails for each cage. The quails were fed with a poisonous feedstuff twice a day at the same feeding amount for a long time, with an average feeding amount of 20 g per day for each quail. Since the feeding started, the body weight was recorded for quails in each treatment; the number of eggs produced on day 40 to 48 was counted; the eggs produced on day 45 to 50 were collected and hatched in a poultry-specific hatcher separately for each treatment; and the stillbirth rate was calculated. In this example, the endpoint effect data were body weight, egg production and stillbirth rate.

Step 2: Analysis of the Data Properties

The data type, monotonicity, normality and homogeneity of variance were analyzed separately for the endpoint effect data according to existing techniques to determine the properties of the data.

In this example, for the acetamiprid test group, the body weight data were consistent with the third type: continuous variables, with normality and homogeneity of variance, but without monotonicity; the egg production data were consistent with the fifth type: continuous variables, without monotonicity and normality; and the stillbirth rate data were consistent with the second type: binary variables, without monotonicity.

For the MCPA test group, the body weight data were consistent with the fourth type: continuous variables, with normality, but without monotonicity and homogeneity of variance; the egg production data were consistent with the first type: continuous variables, with monotonicity; and the stillbirth rate data were consistent with the first type: binary variables, with monotonicity. The above data properties were determined to establish an analysis method.

Step 3: Construction of a Hypothesis Testing Model for NOEC Analysis Based on the Data Properties and Screening of Treatment Groups that had No Significant Difference with the Control Group

The hypothesis testing model had the following specific steps:

A trend test model was adopted when the data were consistent with the first type. For the egg yield data and the stillbirth rate data of the MCPA test group, Jonckheere-Terpstra test was adopted.

A non-parametric paired comparison test model was adopted when the data were consistent with the second type. For the stillbirth rate data of the acetamiprid test group, Fisher's exact test based on Bonferroni-Holm correction was adopted.

A paired comparison test model was adopted when the data were consistent with the third type. For the body weight data of the acetamiprid test group, Dunnett's test was adopted.

A heteroscedasticity paired comparison test model was adopted when the data were consistent with the fourth type. For the body weight data of the MCPA test group, Tamhane-Dunnett test was adopted.

A non-parametric paired comparison test model was adopted when the data were consistent with the fifth type. For the egg production data of the acetamiprid test group, Mann-Whitney test based on Bonferroni-Holm correction was adopted.

The above test models were inspected with SPSS software, and the effort involving Bonferroni-Holm correction was done by analysts themselves.

With the above models, the NOEC revealed by each endpoint effect was determined according to the statistical significance value from each test model. For acetamiprid, based on the body weight, egg production and stillbirth rate, NOEC for chronic toxicity in quails was calculated as 130 mg/kg_{feedstuff}. For MCPA, NOEC for chronic toxicity in quails was calculated as 25 mg/kg_{feedstuff} based on the body weight; NOEC for chronic toxicity in quails was calculated as 500 mg/kg_{feedstuff} based on the egg production; and NOEC for chronic toxicity in quails was calculated as 25 mg/kg_{feedstuff} based on the stillbirth rate.

TABLE 2
The effect of acetamiprid and MCPA on the body weight,
egg production and stillbirth rate of quails

| | Mass | Body | Egg | Stillbirth |
|---|---|---|---|---|
| | concentration | weight | production | rate |
| Chemical | (mg/L_{feedstuff}) | (g) | (eggs) | (%) |
| Acetamiprid | 0 | 152.3 a | 0.97 a | 23.9 a |
| | 0.5 | 150.9 a | 0.87 a | 23.4 a |
| | 2 | 156.7 a | 0.86 a | 16.7 a |
| | 7 | 153.6 a | 0.87 a | 25.7 a |
| | 30 | 151.9 a | 0.89 a | 26.4 a |
| | 130 | 145.4 a | 0.86 a | 21.9 a |
| MCPA | 0 | 151.4 ab | 0.98 a | 19.3 a |
| | 1.25 | 148.5 ab | 0.97 a | 22.4 a |
| | 6.25 | 155.3 ab | 0.96 a | 23.2 ab |
| | 25 | 150.7 ab | 0.96 a | 21.2 a |
| | 125 | 145.2 b | 0.95 a | 26.4 b |
| | 500 | 159.5 a | 0.94 a | 37.3 b |

Step 4: Analysis and Determination of NOEC

Given the results of the test and analysis for each endpoint effect at the test concentrations, NOEC of acetamiprid was determined as 130 mg/kg_{feedstuff}; and NOEC of MCPA was determined as 25 mg/kg_{feedstuff}.

Validation: The test was conducted once again with acetamiprid at a concentration of 125 mg/L and a blank control, the body weight, egg production and stillbirth rate were investigated for quails, and there was no significant difference.

The test was conducted once again with MCPA at a concentration of 20 mg/L and a blank control, the body weight was investigated for quails, and there was no significant difference between the two groups.

It can be seen from the above examples that the analysis method provided in the present invention can ensure the efficiency and accuracy of the analysis by classifying the endpoint effect data of a chronic toxicity test and using different trend test models for different types of data.

The above descriptions are merely preferred implementations of the present invention. It should be noted that a person of ordinary skill in the art may further make several improvements and modifications without departing from the principle of the present invention, but such improvements and modifications should be deemed as falling within the protection scope of the present invention.

What is claimed is:

1. A method for analyzing a non-observed effect concentration (NOEC) of a test chemical on a test organism, comprising the following steps:
1) conducting a chronic toxicity test on the test organism with the test chemical at different concentrations, and conducting assays to obtain several sets of endpoint effect data;
2) classifying each set of the several sets of endpoint effect data obtained in step 1) into one of a plurality of types of data including: type a data, type b data, type c data, type d data, and type e data, wherein, the type a data have monotonicity; the type b data are binary variables that do not have monotonicity; the type c data are continuous variables that do not have monotonicity, and the type c data conform to the normal distribution and homogeneity of variance; the type d data are continuous variables that do not have monotonicity, but the type d data only conform to the normal distribution; and the type e data are continuous variables that do not have monotonicity, but the type e data do not conform to the normal distribution; and
3) for each type of data in the plurality of types of data classified in step 2), constructing hypothesis testing models, and according to statistical significance values from hypothesis testing models, among each set of the several sets of endpoint effect data classified into a particular data type, adopting a highest concentration of the test chemical that does not produce a significant effect as NOEC within the particular data type; and among the highest concentrations of the test chemical adopted for each type of data in the plurality of data types, adopting a smallest concentration as NOEC of the test chemical on the test organism;
wherein, a trend test model is adopted when the data are consistent with the type a data;
a non-parametric paired comparison test model is adopted when the data are consistent with the type b data;
a paired comparison test model is adopted when the data are consistent with the type c data;
a heteroscedasticity paired comparison test model is adopted when the data are consistent with the type d data; and
a non-parametric paired comparison test model is adopted when the data are consistent with the type e data.

2. The analysis method according to claim 1, wherein the test organism comprises animals.

3. The analysis method according to claim 2, wherein the animals comprise insects and birds.

4. The analysis method according to claim 3, wherein, when the test organism is Trichogramma, the several sets of endpoint effect data comprise one or more of egg yield, emergence rate, adult survival time, parasitism rate, mortality rate and hatching rate.

5. The analysis method according to claim 4, wherein, when the test organism is Trichogramma, conducting the chronic toxicity test comprises dipping egg cards into the test chemical at different concentrations.

6. The analysis method according to claim 3, wherein, when the test organism is quail, the several sets of endpoint effect data comprise one or more of 14-day survival rate, embryo survival rate, hatching rate, emergence rate, feeding amount, body weight, average daily egg production, average egg production and stillbirth rate.

7. The analysis method according to claim 6, wherein, when the test organism is quail, conducting the chronic toxicity test comprises feeding the quail with a feedstuff admixed with the test chemical.

8. The analysis method according to claim 1, wherein, the different concentrations in step 1) comprise 4 to 10 different concentrations.

9. The analysis method according to claim 1, wherein, the trend test model comprises Jonckheere-Terpstra test; the non-parametric paired comparison test model comprises Fisher's exact test based on Bonferroni-Holm correction; the paired comparison test model comprises Dunnett's test; the heteroscedasticity paired comparison test model comprises Tamhane-Dunnett test; and the non-parametric paired comparison test model comprises Mann-Whitney test based on Bonferroni-Holm correction.
